(19) United States
(12) Patent Application Publication
(10) Pub. No.: US 2026/0216395 A1
(43) Pub. Date: Jul. 30, 2026

(54) DISINFECTING DEVICE WITH INDICATOR

(71) Applicant: Becton, Dickinson and Company, Franklin Lakes, NJ (US)

(72) Inventors: Ravikumar Mohan, Chennai (IN); Rakshitha K R, Bangalore (IN); Prabhath Kakaraparthi, Nellore, Andhra Pradesh (IN); Rahul Malviya, Bangalore, Karnataka (IN)

(73) Assignee: Becton, Dickinson and Company, Franklin Lakes, NJ (US)

(21) Appl. No.: 19/146,829

(22) PCT Filed: Feb. 3, 2023
(86) PCT No.: PCT/US2023/061936

§ 371 Date: Jul. 9, 2025

(51) Int. Cl. A61L 2/28; A61L 2/18; A61L 103/06

(52) U.S. Cl. CPC A61L 2/28; A61L 2/18; A61L 2103/06

(57) ABSTRACT

Provided herein is a disinfecting device including: a substrate having a first side and an opposing second side and including a first material; and an indicator region embedded in the substrate, where the indicator includes a solvatochromatic material coated with a masking material embedded in the first side of the substrate.

### BACKGROUND OF THE INVENTION

### Field of the Invention

The present invention is directed to a disinfecting device having an indicator.

### Description of Related Art

Disinfecting devices may be used by health care professionals (HCPs) to disinfect a region prior to proceeding with a medical activity. For example, the skin of a patient and/or a medical device may be disinfected before a medical-related procedure. Non-limiting examples of disinfecting devices include alcohol swabs.

HCPs performing the disinfecting activity must exercise care to sufficiently disinfect the region. However, it is often unclear to the HCP whether sufficient cleaning has occurred to disinfect the region.

### SUMMARY OF THE INVENTION

Provided herein is a disinfecting device including: a substrate having a first side and an opposing second side and including a first material; and an indicator region embedded in the substrate, where the indicator includes a solvatochromatic material coated with a masking material embedded in the first side of the substrate.

Also provided herein is a method for disinfecting a region including rubbing the disinfecting device described herein against a region until the solvatochromatic material changes from a first color to a second color.

Also provided herein is a method of manufacturing a disinfecting device including: embedding a solvatochromatic material in a substrate including a first material; coating the solvatochromatic material on a first side with a masking material; and sealing the substrate in a pouch filled with a disinfecting solvent.

Other non-limiting embodiments of the invention will be set forth in the following numbered clauses:

Clause 1: A disinfecting device, comprising: a substrate having a first side and an opposing second side and comprising a first material; and an indicator region embedded in the substrate, wherein the indicator region comprises a solvatochromatic material coated with a masking material embedded in the first side of the substrate.

Clause 2: The disinfecting device of clause 1, wherein the solvatochromatic material is coated by a transparent material embedded in the second side of the substrate.

Clause 3: The disinfecting device of clause 1 or 2, wherein the first material comprises an absorbent material.

Clause 4: The disinfecting device of any of clauses 1-3, wherein a solvent is absorbed into the first material.

Clause 5: The disinfecting device of clause 4, wherein the solvent comprises a polar solvent.

Clause 6: The disinfecting device of clause 4 or 5, wherein the masking material is insoluble in the solvent.

Clause 7: The disinfecting device of any of clauses 4-6, wherein the solvent is a disinfecting solvent.

Clause 8: The disinfecting device of any of clauses 4-7, wherein the solvatochromatic material is encased so as to be free from contact with the solvent.

Clause 9: The disinfecting device of any of clauses 1-8, wherein the first material comprises a porous absorbent material.

Clause 10: The disinfecting device of any of clauses 1-9, wherein the masking material comprises a wax.

Clause 11: The disinfecting device of clause 10, wherein the wax comprises a natural beeswax.

Clause 12: The disinfecting device of any of clauses 1-11, wherein the masking material is opaque.

Clause 13: The disinfecting device of any of clauses 4-12, wherein the solvatochromatic material changes colors when contacted by the solvent.

Clause 14: The disinfecting device of any of clauses 1-13, wherein a thickness of the masking material is selected such that the masking material wears off the disinfecting device to expose the solvatochromatic material after a predetermined number of rubs against a disinfecting region.

Clause 15: The disinfecting device of any of clauses 1-14, sealed in a pouch comprising disinfecting solvent.

Clause 16: A method for disinfecting a region comprising: rubbing the disinfecting device of any of clauses 1-15 against a region until the solvatochromatic material changes from a first color to a second color.

Clause 17: A method of manufacturing a disinfecting device comprising: embedding a solvatochromatic material in a substrate comprising a first material; coating the solvatochromatic material on a first side with a masking material; and sealing the substrate in a pouch filled with a disinfecting solvent.

### BRIEF DESCRIPTION OF THE DRAWINGS

FIG. 1 is a schematic view of a disinfecting device according to non-limiting embodiments described herein;

FIG. 2 is a schematic view of a disinfecting device in use with an “incomplete” indicator according to non-limiting embodiments described herein;

FIG. 3 is a schematic view of a disinfecting device in use with a “complete” indicator according to non-limiting embodiments described herein;

FIG. 4A is a schematic view of a disinfecting device in use with an “incomplete” indicator according to non-limiting embodiments described herein;

FIG. 4B is a schematic view of a disinfecting device in use with a “complete” indicator according to non-limiting embodiments described herein;

FIG. 5 is a schematic view of a disinfecting device sealed in a pouch according to non-limiting embodiments described herein; and

FIG. 6 is a step diagram of a method of manufacturing a disinfecting device according to non-limiting embodiments described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

The present disclosure is directed to a disinfecting device, comprising: a substrate having a first side and an opposing second side and comprising a first material; and an indicator region embedded in the substrate, wherein the indicator region comprises a solvatochromatic material coated with a masking material embedded in the first side of the substrate.

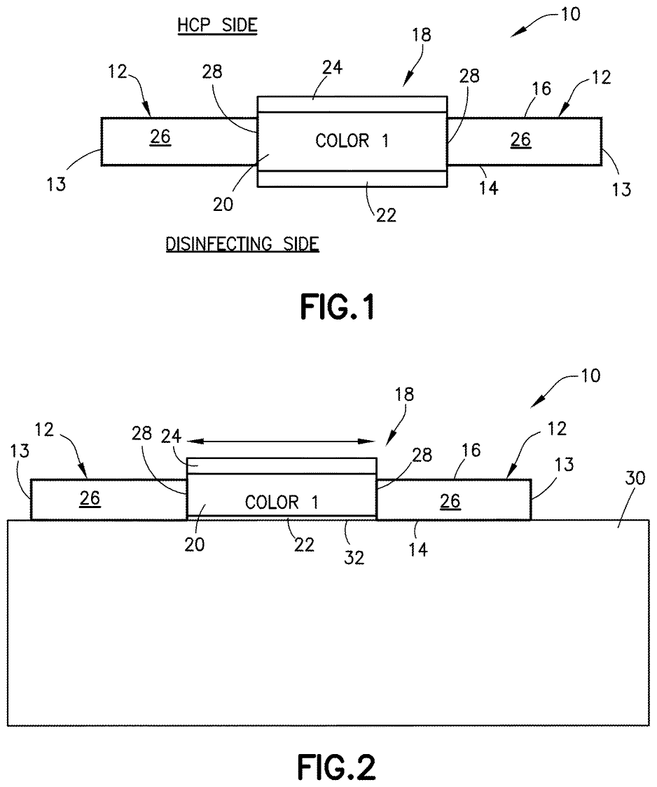

Referring to FIGS. 1-3, a disinfecting device 10 is shown for disinfecting a region. The disinfecting device 10 may comprise a substrate 12 having a first side 14 and an opposing second side 16 and comprising a first material 13. The first side 14 may be the disinfecting side rubbed against the region to disinfect the region. The second side 16 may be the side seen by the user of the disinfecting device 10 when in use to disinfect the region. Because the user of the disinfecting device 10 may commonly be a health care professional (HCP), this second side 16 is referred to as the HCP side.

The first material 13 of the substrate 12 may comprise an absorbent material. For example, the absorbent material may comprise a porous absorbent material, such as cotton gauze, a natural fiber sponge, a porous silica media, a foamed polyurethane or any other polymer, a non-woven polymeric fabric, a hydrogel, or other material suitable for absorbing a liquid, such as the disinfecting solvent described herein.

With continued reference to FIGS. 1-3, the disinfecting device 10 may comprise an indicator region 18 embedded in the substrate 12. The indicator region 18 may be embedded in the substrate 12 by being at least partially surrounded by the substrate 12, such as by being fully surrounded by the substrate 12. The indicator region 18 may comprise a solvatochromatic material 20. The solvatochromatic material 20 may be coated on the side corresponding to the first side 14 of the substrate 12 by a masking material 22. The solvatochromatic material 20 may be coated on the side corresponding to the second side 16 of the substrate 12 by a transparent material 24. Thus, as shown in FIGS. 1-3, the first side 14 of the substrate 12 may be adjacent to, such as in direct contact with, the masking material 22, and the second side 16 of the substrate 12 may be adjacent to, such as in direct contact with, the transparent material 24.

The solvatochromatic material 20 may be any suitable material that changes colors in response to coming into contact with a disinfecting solvent used with the disinfecting device 10. The solvatochromatic material 20 may be a chromatic dye exhibiting a color detectable by the human eye. The solvatochromatic material 20 may be a solvatochromatic polymer. Non-limiting examples of the solvatochromatic material 20 include a conjugated polydiacetylene (PDA) polymer, such as a PDA polymer derived from 10,12-pentacosadiynoic acid (PCDA) and/or 10,12-tricosadiynoic acid. The solvatochromatic material 20 may comprise a compound having diacetylene chemistry. The solvatochromatic material may comprise a spiropyran, an azobenzene, or other similar compound. The solvatochromatic material may comprise poly(phenylene vinylene), such as poly(p-phenylene vinylene) (PPV), polythiophene, polyphenylene, polyacetylene, poly(p-naphthalene vinylene), poly(2,5-pyridyl vinylene), and derivatives thereof. This PDA polymer may be suitable for use in disinfecting devices used for medical applications, as it is biocompatible and found safe for use with humans. In this example, the PDA polymer may be a blue color if not in contact with the disinfecting solvent, and may change to a red color when coming into contact with the disinfecting solvent, such as isopropyl alcohol (IPA). The solvatochromatic material 20 may be a conjugated polymer, as the bond conjugation may be a mechanism by which the solvatochromatic material 20 changes color when exposed to the disinfecting solvent.

It will be appreciated that any suitable solvatochromatic material 20 may be used that exhibits a change in color detectable by the human eye in response to contacting the disinfecting solvent. The change in color may be from a first color to a second color. The first color and the second color may be different colors or different shades of the same color. The solvatochromatic material 20 may be selected based on the disinfecting solvent used, such that the solvatochromatic material 20 exhibits a color change response when contacted with the disinfecting solvent used.

The disinfecting solvent may comprise any solvent suitable for disinfecting a region and interacting with the solvatochromatic material 20 to change the color thereof. Non-limiting examples of disinfecting solvents include IPA, chlorhexidine digluconate (CHG), chloroxylenol (PCMX), povidone-iodine (PVP), or some mixture thereof. The solvent may be a polar solvent.

With continued reference to FIGS. 1-3, the solvatochromatic material 20 may be coated on the side corresponding to the second side 16 of the substrate 12 by the transparent material 24. The transparent material 24 may allow the user (e.g., the HCP) to see therethrough such that the solvatochromatic material 20 beneath the transparent material 24 is at least partially visible to the user. The transparent material 24 being transparent allows the user to see the change in color (or lack thereof) of the solvatochromatic material 20 which, as described herein, may indicate that the disinfecting device 10 has suitably disinfected the region. Any suitable transparent material 24 may be used. Non-limiting examples of transparent materials comprise transparent waxes, such as paraffin waxes, natural waxes such as carnauba wax, candelilla wax, vegetable oil waxes, wax esters, synthetic olefinic waxes, and the like. Natural beeswax may be a suitable wax used for the transparent materials 24.

With continued reference to FIGS. 1-3, the solvatochromatic material 20 may be coated on the side corresponding to the first side 14 of the substrate 12 by the masking material 22. The masking material 22 may coat an entire surface of the solvatochromatic material 20 on the first side 14 so that no solvatochromatic material 20 is exposed for possible contact with the disinfecting solvent prior to the masking material 22 being later worn away. The masking material 22 may be any soft and/or malleable material having a suitable coefficient of friction such that the masking material 22 wears away when the disinfecting device 10 is rubbed against the region to be disinfected. The masking material 22 may rub against the region to be disinfected, which causes the masking material 22 to wear away. The masking material 22 may also be a biocompatible material suitable for contact with humans, as the region to be disinfected may a part of the human body or may come into contact with the human body.

Non-limiting examples of masking materials 22 comprise waxes. The wax may be natural beeswax, which may have antibiotic and/or hypoallergenic properties. The wax may be compounded with an oil plasticizer. The masking material 22 may be opaque, such that the color of the solvatochromatic material 20 beneath the masking material 22 is not visible while the masking material 22 covers the solvatochromatic material 20. The wax may be colored to the point of being opaque. The masking material 22 may be non-polar.

The masking material 22 may be insoluble in the disinfecting solvent. The insolubility of the masking material 22 with the disinfecting solvent may ensure that the masking material 22 is not removed by interaction with the disinfecting solvent, but is instead mechanically (by the friction forces) removed by rubbing against the region being disinfected.

The masking material 22 may form a layer having a thickness over the solvatochromatic material 20. The thickness of the masking material 22 may be selected such that the masking material 22 wears off the disinfecting device 10 under certain conditions. For example, the thickness may be selected such that the masking material 22 wears off the disinfecting device 10 after an effective number of rubs and/or after an effective rubbing time. The thickness may range, for example, from 15-150 μm. For example, the effective number of rubs may comprise at least 5 rubs, at least 8 rubs, at least 10 rubs, or at least 15 rubs. For example, the effective rubbing time may be at least 5 seconds, at least 10 seconds, at least 15 seconds rubs, or at least 20 seconds. Factors affecting the thickness of the masking material may include, for example, the material of the masking material 22 (e.g., the hardness, viscosity, melting temperature and other properties thereof), the material of the disinfecting region (e.g., human skin, metal medical device, polymeric medical device, etc.) (e.g., the hardness and other properties thereof), coefficient of friction, and the like. The viscosity of the masking material 22 may be controlled to the desired viscosity by adding a suitable amount of plasticizer thereto.

Upon the wearing away of the masking material 22, the solvatochromatic material 20 beneath the masking material 22 may be exposed to the disinfecting solvent, which interaction may cause the solvatochromatic material 20 to change from the first color to the second color. This color change may indicate to the user that disinfecting has been suitably completed by the disinfecting device 10.

With continued reference to FIGS. 1-3, the first material 13 may hold absorbed solvent 26. The absorbed solvent 26 absorbed by the first material 13 may be released during use of the disinfecting device 10 by the first material 13 being rubbed against the disinfecting region, which may squeeze at least a portion of the absorbed solvent 26 from being trapped in the first material 13 to the disinfecting region, in order to effect disinfection of the disinfecting region.

With continued reference to FIGS. 1-3, the solvatochromatic material 20 may be encased so as to be free from contact with the disinfecting solvent until after the masking material 22 is rubbed from disinfecting device 10. The masking material 22 and the transparent material 24 may function as barriers to prevent the disinfecting solvent from contacting the solvatochromatic material 20 until the masking material 22 is rubbed away. The disinfecting device may comprise barriers 28 between the first material 13 comprising absorbed solvent 26 and the solvatochromatic material 20 to prevent the disinfecting solvent from contacting the solvatochromatic material 20 until the masking material 22 is rubbed away. The barriers 28 may comprise the same materials as the masking material 22 and the transparent material 24, and when the masking material 22 and the transparent material 24 are applied to the solvatochromatic material 20, they may also flow to completely encapsulate the solvatochromatic material 20 to form the barriers 28. The solvatochromatic material 20 may be polymerized so as to be impenetrable to wax but may be penetrated by the disinfecting solvent.

Referring to FIGS. 2-4B, the disinfecting device 10 in use is shown. The disinfecting device 10 may be used to disinfect the surface 32 of an object 30. The object 30 may be a human or animal, such as the skin thereof being disinfected by the disinfecting device 10. The object 30 may be a medical device. For example the medical device may be a catheter component, a connector, or the like. The disinfecting device 10 may be rubbed against the surface 32 of the object 30 with the masking material 22 and the first side 14 of the substrate 12 in contact with the surface 32. By the rubbing of the disinfecting device 10 on the surface 32, the masking material 22 may gradually wear away due to the friction forces.

In FIG. 2, the disinfecting device 10 is in use to disinfect the surface 32, and the indicator region 18 exhibits an “incomplete” indicator. The color of the solvatochromatic material 20 prior to use of the disinfecting device 10 is “Color 1” (see FIG. 1). FIG. 2 shows an “incomplete” indictor because the color of the solvatochromatic material 20 is still “Color 1.” This is because the solvatochromatic material 20 has not yet been contacted with any disinfecting solvent being used to disinfect the surface 32. The solvatochromatic material 20 is still encased by the transparent material 24, the barriers 28, and the masking material 22 to prevent contact between the disinfecting solvent and the solvatochromatic material 20. Compared to FIG. 1, approximately half of the masking material 22 has worn away in FIG. 2 due to the rubbing between the disinfecting device 10 and the surface 32 (as evidenced by the thickness of the masking layer 22 being about half as thick in FIG. 2 compared to FIG. 1). Since the solvatochromatic material 20 still exhibits “Color 1” only incomplete disinfection has been effected.

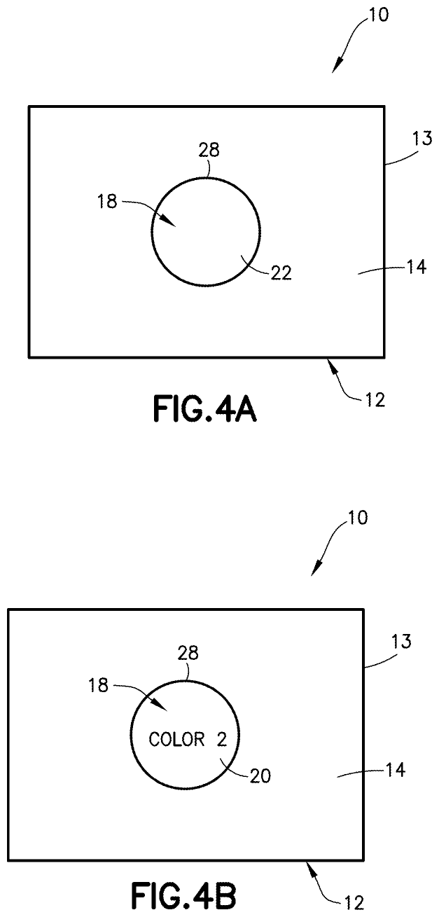

FIG. 4A shows the disinfecting device 10 of FIG. 2 from the perspective of the first side 14 thereof. As shown, the indicator region 18 may be embedded in the substrate 12. For the “incomplete” indicator, the masking material 22 may still fully cover the solvatochromatic material 20 such that none of the solvatochromatic material 20 is exposed to contact with the disinfecting solvent. In the embodiments in which the masking material 22 is opaque, no color of the solvatochromatic material 20 (e.g., Color 1 or Color 2) is visible, but only the color of the opaque masking material 22. In embodiments in which the masking material 22 is transparent, Color 1 of the solvatochromatic material 20 may be visible through the masking material 22 since the solvatochromatic material 20 has not contacted any disinfecting solvent.

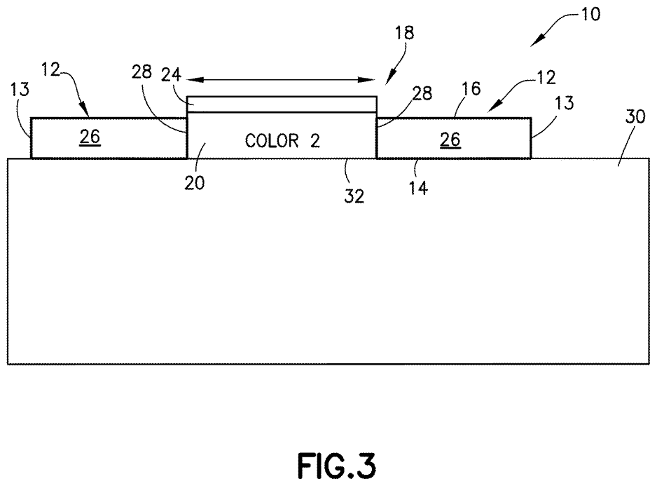

In FIG. 3, the disinfecting device 10 is in use to disinfect the surface 32, and the indicator region 18 exhibits a “complete” indicator. The color of the solvatochromatic material 20 prior to use of the disinfecting device 10 is “Color 1” (see FIG. 1). FIG. 3 shows a “complete” indictor because the color of the solvatochromatic material 20 has changed to “Color 2.” This is because the solvatochromatic material 20 has contacted disinfecting solvent being used to disinfect the surface 32. The solvatochromatic material 20 is still encased by the transparent material 24 and the barriers 28, but not by the masking material 22, which has been worn by rubbing against the surface 32. Compared to FIG. 1, a section of the masking material 22 has worn away in FIG. 3 due to the rubbing between the disinfecting device 10 and the surface 32 (as evidenced by the thickness of the masking layer 22 disappearing in FIG. 3 compared to FIG. 1) to expose at least a portion of the solvatochromatic material 20 to disinfecting solvent. Since the solvatochromatic material 20 now exhibits “Color 2” complete disinfection has been effected.

FIG. 4B shows the disinfecting device 10 of FIG. 3 from the perspective of the first side 14 thereof. As shown, the indicator region 18 may be embedded in the substrate 12. For the “complete” indicator, the masking material 22 may be at least partially worn away to the extent that at least a portion (e.g., the entire region of solvatochromatic material 20 in FIG. 4B) of the solvatochromatic material 20 is exposed to contact with the disinfecting solvent. Because solvatochromatic material 20 is exposed to the disinfecting solvent (and visually to the user and/or patient/object), the portion of the solvatochromatic material 20 may change from Color 1 to Color 2, indicating complete disinfection of the region being disinfected.

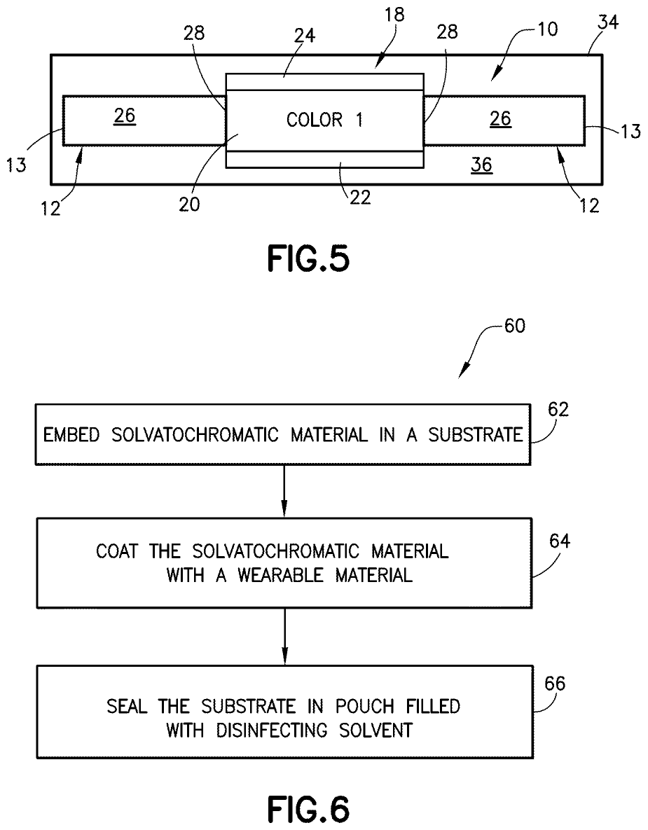

Referring to FIG. 5 a disinfecting device 10 sealed in a pouch 34 is shown. The disinfecting device 10 as described herein may be inserted into the pouch 34 containing liquid disinfecting solvent 36. The pouch 34 may be sealed shut. The first material 13 of the disinfecting device 10 may absorb at least a portion of the disinfecting solvent 36 in the pouch 34 to form absorbed solvent 26. The pouch 34 may be any suitable pouch for maintaining the disinfecting device 10 having the absorbed solvent 26. The pouch 34 may be an aluminum pouch, a multilayer polymeric pouch, or a pouch made from any other gas barrier capable materials.

The disinfecting device 10 may be used to disinfect a region by opening the sealed pouch 34, removing the disinfecting device 10 therefrom, and rubbing the disinfecting device 10 on the region (first side 14 and masking material 22 in contact with the region) to be disinfected until the solvatochromatic material 20 changes from the first color to the second color.

Referring to FIG. 6, a method 60 is shown for manufacturing the disinfecting device described herein. At a step 62, the solvatochromatic material may be embedded in a substrate comprising the first material. The solvatochromatic material may be embedded in the substrate using any suitable technique, such as a suitable printing technique. For example, the solvatochromatic material may be embedded in the substrate by gravure printing.

At a step 64, the masking material may be coated onto the first side of the solvatochromatic material. The masking material may be coated onto the solvatochromatic material using any suitable technique, such as a suitable printing technique. For example, the masking material may be coated onto the solvatochromatic material by gravure printing. The transparent material may be coated on to the second side of the solvatochromatic material using any suitable technique, such as the techniques disclosed in connection with applying the masking material.

At a step 66, the substrate (containing the solvatochromatic material and the masking material) may be inserted into the pouch comprising the disinfecting solvent, and the pouch may be sealed closed.

Although the present disclosure has been described in detail for the purpose of illustration based on what is currently considered to be the most practical and preferred embodiments, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

The invention claimed is:

1. A disinfecting device, comprising:
a substrate having a first side and an opposing second side and comprising a first material; and
an indicator region embedded in the substrate, wherein the indicator region comprises a solvatochromatic material coated with a masking material embedded in the first side of the substrate.

2. The disinfecting device of claim 1, wherein the solvatochromatic material is coated by a transparent material embedded in the second side of the substrate.

3. The disinfecting device of claim 1, wherein the first material comprises an absorbent material.

4. The disinfecting device of claim 1, wherein a solvent is absorbed into the first material.

5. The disinfecting device of claim 4, wherein the solvent comprises a polar solvent.

6. The disinfecting device of claim 4, wherein the masking material is insoluble in the solvent.

7. The disinfecting device of claim 4, wherein the solvent is a disinfecting solvent.

8. The disinfecting device of claim 4, wherein the solvatochromatic material is encased so as to be free from contact with the solvent.

9. The disinfecting device of claim 1, wherein the first material comprises a porous absorbent material.

10. The disinfecting device of claim 1, wherein the masking material comprises a wax.

11. The disinfecting device of claim 10, wherein the wax comprises a natural beeswax.

12. The disinfecting device of claim 1, wherein the masking material is opaque.

13. The disinfecting device of claim 4, wherein the solvatochromatic material changes colors when contacted by the solvent.

14. The disinfecting device of claim 1, wherein a thickness of the masking material is selected such that the masking material wears off the disinfecting device to expose the solvatochromatic material after a predetermined number of rubs against a disinfecting region.

15. The disinfecting device of claim 1, sealed in a pouch comprising disinfecting solvent.
